(19) United States
(12) Patent Application Publication
(10) Pub. No.: US 2022/0242725 A1
(43) Pub. Date: Aug. 4, 2022

(54) MANUFACTURING METHODS FOR DUAL PORE SENSORS

(71) Applicant: Applied Materials, Inc., Santa Clara, CA (US)

(72) Inventors: Joseph R. JOHNSON, Redwood City, CA (US); Roger QUON, Rhinebeck, NY (US); Archana KUMAR, Mountain View, CA (US); Ryan Scott SMITH, Clifton Park, NY (US); Jeremiah HEBDING, East Berne, NY (US); Raghav SREENIVASAN, Fremont, CA (US)

(21) Appl. No.: 17/617,151

(22) PCT Filed: Apr. 15, 2020
(86) PCT No.: PCT/US2020/028268

§ 371 Date: Dec. 7, 2021

Related U.S. Application Data
(60) Provisional application No. 62/858,722, filed on Jun. 7, 2019.

(51) Int. Cl. B82B 3/00; B81C 1/00; B82Y 15/00; B82Y 40/00

(52) U.S. Cl. CPC B82B 3/0019; B81C 1/00087; B82Y 15/00; B81B 2207/056; B81C 2201/0133; B81B 2201/0214; B81B 2203/0127; B82Y 40/00

(57) ABSTRACT

Embodiments of the present disclosure provide methods of forming solid state dual pore sensors which may be used for biopolymer sequencing and dual pore sensors formed therefrom. In one embodiment, a method of forming a dual pore sensor includes providing a pattern in a surface of a substrate. Generally, the pattern features two fluid reservoirs separated by a divider wall. The method further includes depositing a layer of sacrificial material into the two fluid reservoirs, depositing a membrane layer, patterning two nanopores through the membrane layer, removing the sacrificial material from the two fluid reservoirs, and patterning one or more fluid ports and a common chamber.

### BACKGROUND

### Field

Embodiments herein relate to flow cells to be used with solid-state nanopore sensors and methods of manufacturing thereof.

### Description of the Related Art

Solid-state nanopore sensors have emerged as a low-cost, easily transportable, and rapid processing biopolymer, e.g., DNA or RNA, sequencing technology. Solid-state nanopore sequencing of a biopolymer strand typically incudes translocating a biopolymer strand through one or more nanoscale sized openings each having a diameter between about 0.1 nm and about 100 nm, i.e., a nanopore. In a single pore sensor, a nanopore is disposed through a membrane layer which separates two conductive fluid reservoirs. The biopolymer strand to be sequenced, e.g., a characteristically negatively charged DNA or RNA strand, is introduced into one of the two conductive fluid reservoirs and is then drawn through the nanopore by providing an electric potential therebetween. As the biopolymer strand travels through the nanopore the different monomer units thereof, e.g., protein bases of a DNA or RNA strand, occlude different percentages of the nanopore thus changing the ionic current flow therethrough. The resulting current signal pattern can be used to determine the sequence of monomer units in the biopolymer strand, such as the sequence of proteins in a DNA or RNA strand. Generally, single pore sensors lack a mechanism for slowing the rate of translocation of the biopolymer strand through the nanopore while still providing sufficient electrical potential between the two reservoirs to optimize the signal to noise ratio in the resulting current signal pattern.

Beneficially, dual pore sensors provide a mechanism for controlling the rate of translocation of a biopolymer strand by co-capturing the biopolymer strand in the two nanopores thereof. A typical dual pore sensor features two fluid reservoirs separated by a wall, a common fluid chamber, and a membrane separating the common fluid chamber from each of the fluid reservoirs, the membrane layer having the two nanopores disposed therethrough. A biopolymer strand to be sequenced travels from the first fluid reservoir to the common chamber and from the common chamber to the second fluid reservoir through a second nanopore. Desirably the two nanopores are positioned close enough to one another to allow for co-capture of the biopolymer strand. When the biopolymer strand is co-captured by both of the nanopores, competing electric potentials are applied across each of the nanopores to create a “tug-of-war” where the opposite ends of the biopolymer strand are pulled in opposite directions of travel. Beneficially, the difference between the competing electric potentials can be adjusted to control the rate of translocation of the biopolymer strand through the nanopores and thus the resolution of the electrical signal current signal pattern or patterns resulting therefrom.

Often, dual nanopore sensors are formed using two substrates. Typically, the first substrate is formed of an amorphous non-monocrystalline material, such as glass, which is patterned to form the first and second fluid reservoirs having the wall disposed therebetween. The second substrate is formed of monocrystalline silicon and a multi-layer stack comprising the membrane layer is formed on a surface thereof. The membrane layer of the second substrate is then anodically bonded to the patterned surface of the first substrate, the silicon substrate is removed from the multi-layer stack, and an opening is etched into the multilayer stack to form the common chamber. The nanopores are then formed through respective portions of the membrane layer disposed on either side of the wall using a focused ion beam (FIB) drilling process.

Unfortunately, the manufacturing methods described above are generally incompatible with the high volume manufacturing, quality, repeatability, and cost requirements needed to move dual pore sensors out of the R&D lab and into the public market. Further, the manufacturing methods described above generally limit the minimum spacing between the two nanopores to about 550 nm which thus limits the ability of dual pore sensors formed therefrom to sequence relativity shorter biopolymer strands.

Accordingly, there is a need in the art for improved methods of forming dual pore sensors and improved dual pore sensors formed therefrom.

### SUMMARY

Embodiments of the present disclosure provide solid state dual pore sensors which may be used for biopolymer sequencing, and methods of manufacturing the same.

In one embodiment, a method of forming a dual pore sensor includes providing a pattern in a surface of a substrate. Generally, the pattern features two fluid reservoirs separated by a divider wall. The method further includes depositing a layer of sacrificial material into the two fluid reservoirs, depositing a membrane layer, patterning two nanopores through the membrane layer, removing the sacrificial material from the two fluid reservoirs, and patterning one or more fluid ports and a common chamber.

In another embodiment, a dual pore sensor features a substrate having a patterned surface comprising two recessed regions spaced apart by a divider wall and a membrane layer disposed on the patterned surface. The membrane layer, the divider wall, and one or more surfaces of each of the two recessed regions collectively define a first fluid reservoir and a second fluid reservoir. A first nanopore is disposed through a portion of the membrane layer disposed over the first fluid reservoir and a second nanopore is disposed through a portion of the membrane layer disposed over the second fluid reservoir. Herein, opposing surfaces of the divider wall are sloped to each form an angle of less than 90° with a respective reservoir facing surface of the membrane layer.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A is a close up sectional view schematically illustrating a portion of a dual pore sensor formed using one or a combination of the embodiments described herein.

FIG. 1B schematically illustrates an anisotropically etched surface of a silicon substrate.

FIG. 2 is a flow diagram setting forth a method of forming a dual pore sensor, according to one or more embodiments.

FIGS. 3A-3K schematically illustrate various aspects of the results of the method set forth in FIG. 2.

FIG. 3L schematically illustrates an aspect of the results of an alternative embodiment of the method set forth in FIG. 2.

FIGS. 4A-4B schematically illustrate various aspects of the results of an alternative embodiment of the method set forth in FIG. 2.

FIG. 5 is a plan view of a substrate having a plurality of dual pore sensors formed thereon, according to one embodiment.

### DETAILED DESCRIPTION

Embodiments of the present disclosure provide solid state dual pore sensors which may be used for biopolymer sequencing, and methods of manufacturing the same.

Generally, the dual pore sensors described herein are formed by anisotropically etching openings in a monocrystalline silicon substrate or a monocrystalline silicon substrate surface to form at least two fluid reservoirs which are separated from one another by a divider wall disposed therebetween. The width of the barrier wall limits how close the two nanopores of the dual pore sensors may be spaced from one another and is thus determinative of the minimum length of a biopolymer stand that can be co-captured therebetween.

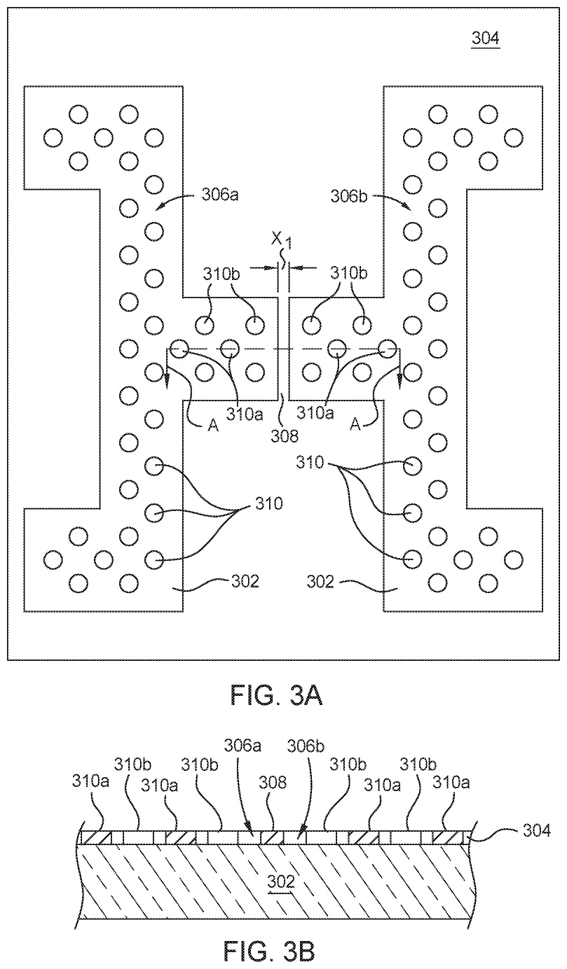

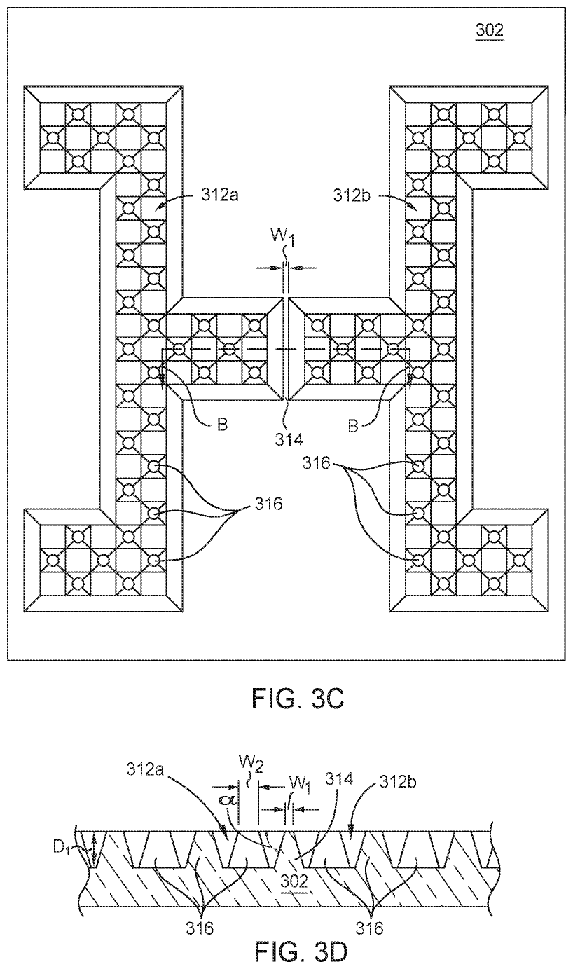

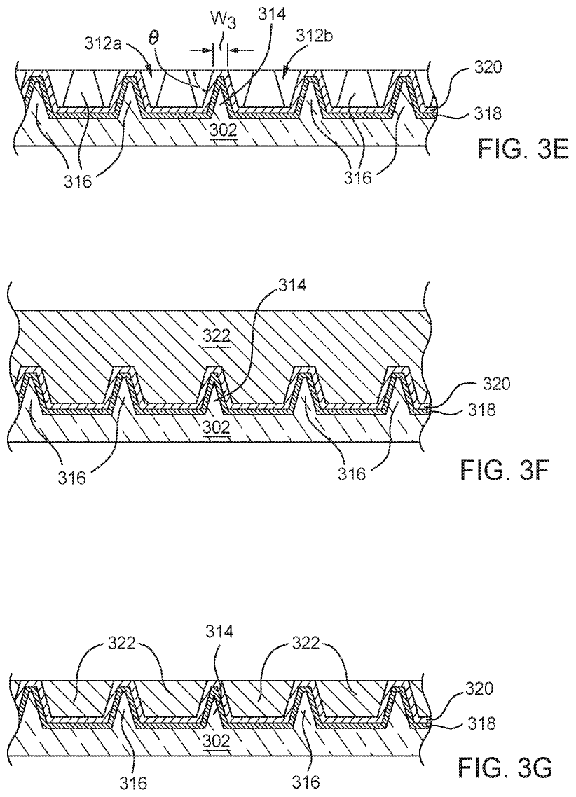

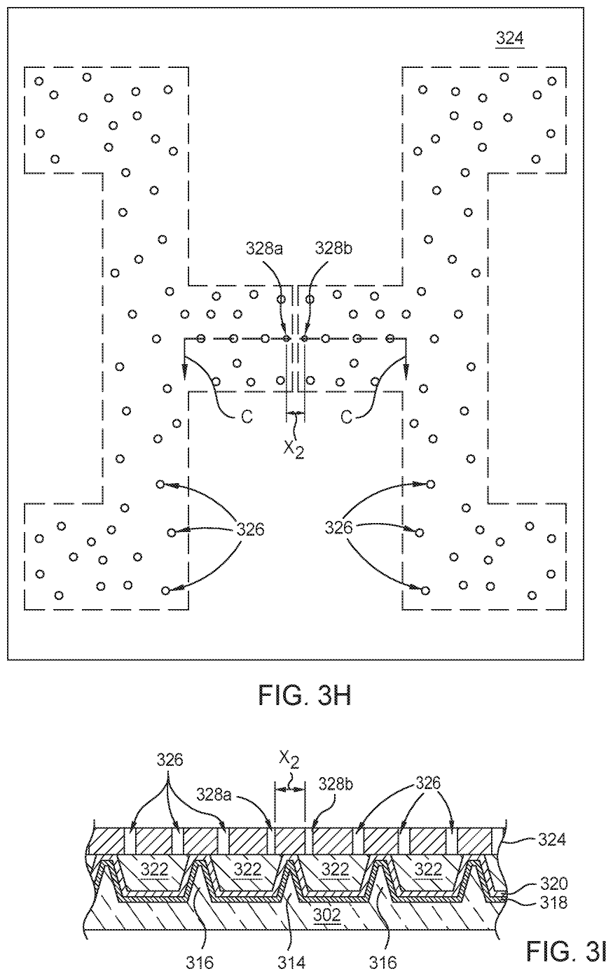

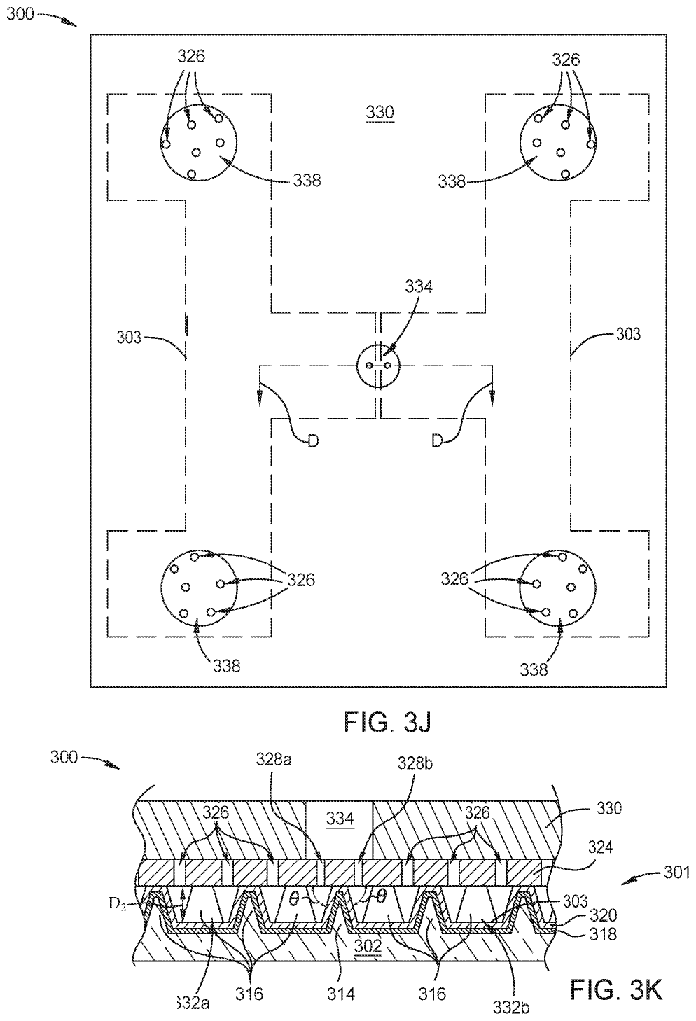

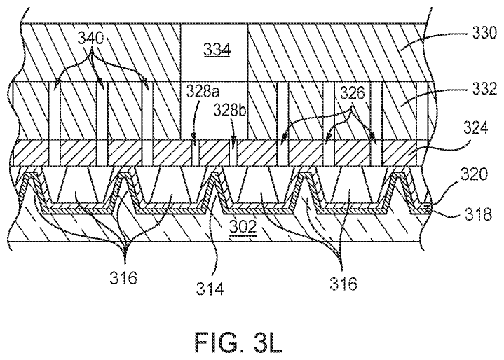

Typically, anisotropically etching the two fluid reservoirs forms a divider wall having a triangular or a trapezoidal shape in cross section, see e.g., the trapezoidal shaped cross section of divider wall 314 shown in FIG. 3D, where the base of the divider wall is wider than the field (upper) surface thereof. In other words, opposing surfaces of the divider wall are sloped to form an angle of less than 90° with a plane of the field surface of the substrate. The sloped surfaces on opposing sides of the divider wall desirably increase stability of the divider wall during manufacturing of the sensor. The added stability allows for the width of the field surface of the divider wall to be narrower, and the fluid reservoirs to be deeper, when compared to a sensor formed from a glass substrate. This is because a divider wall formed in a glass substrate using conventional methods will have vertical sides (i.e., the same wall thickness) along at least a portion the height thereof. Thus, a narrow divider wall formed using conventional methods will undesirably buckle and break as the aspect ratio (height to width ratio) thereof is increased which constrains the manufacturing ability to form narrower walls and deeper reservoirs.

Beneficially, the narrower field surface of the divider walls, made possible by the methods set forth herein, allow for closer spacing of the two nanopores and thus allow for sequencing of shorter biopolymer strands. Further, the deeper reservoirs made possible by the methods set forth herein provide a greater cross sectional area for, and thus provide desirably less resistance to, ionic current flow therethrough.

Examples of suitable substrates which may be used to form the dual pore sensors herein include those commonly used in semiconductor device manufacturing, such as an N-type or P-type doped monocrystalline silicon wafers, or substrates formed undoped monocrystalline silicon, i.e., intrinsic monocrystalline silicon wafers. In some embodiments, the substrate is a doped or undoped silicon wafer having an epitaxial layer of undoped monocrystalline silicon formed thereon. In some embodiments, the substrate features a layered stack of silicon, an electrically insulating material, such as sapphire or a silicon oxide, and silicon, commonly known as a silicon-on-insulator (SOI) substrate or an SOI wafer. When used, undoped silicon substrates, undoped silicon epitaxial layers, and SOI substrates beneficially reduce undesirable parasitic capacitance in a dual pore sensor formed therefrom when compared to a sensor formed of a doped silicon substrate.

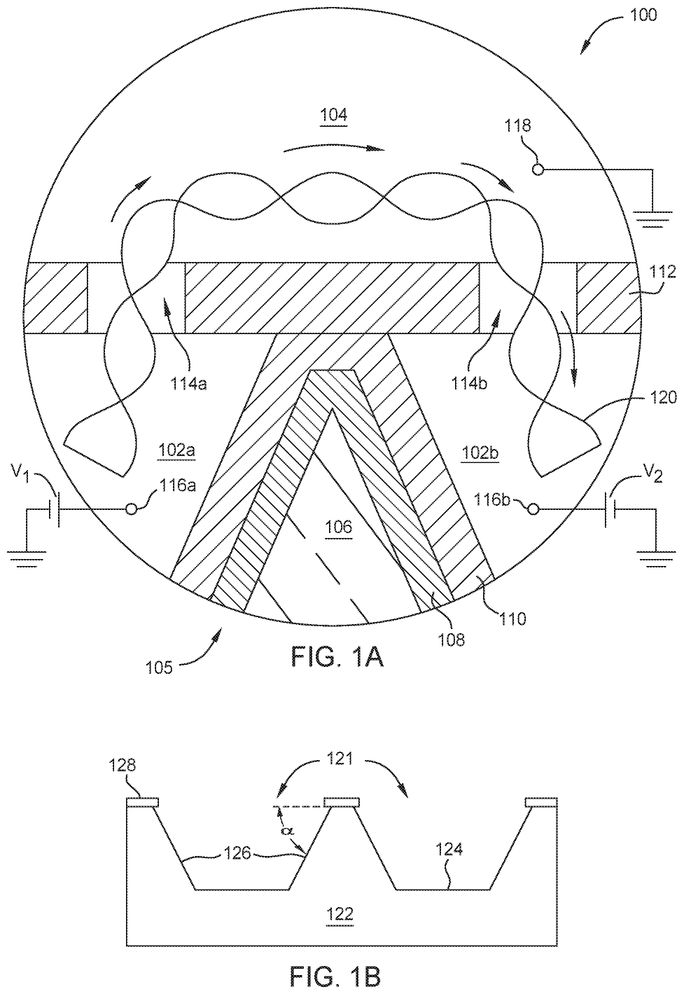

FIG. 1A is a close up sectional view schematically illustrating a portion of a dual pore sensor, formed according to embodiments described herein, which may be used to sequence a biopolymer strand. Here, the dual pore sensor 100 features two fluid reservoirs 102 a, b and a common chamber 104 each of which, in use, have a conductive fluid, such as an electrolytic fluid, disposed therein. The two fluid reservoirs 102 a, b are fluidly isolated from one another by a divider wall 105 disposed therebetween. Here, the divider wall 105 of formed of a continuous portion of an underlying monocrystalline silicon substrate 106 or monocrystalline substrate surface which further includes an oxidized surface layer 108 and a silicon nitride layer 110 disposed on the oxidized surface layer 108. Typically, patterning the underlying monocrystalline silicon substrate 106 forms a triangular or trapezoidal shape in cross section, such as the trapezoidal shaped cross section of the divider wall 314 shown in FIG. 3. Herein, oxidizing the surface to form the oxidized surface layer 108 consumes at least a portion of the silicon from the monocrystalline silicon substrate. Thus, in embodiments where the divider wall is formed to have a trapezoidal shape in cross section, oxidizing the monocrystalline silicon surface may result in the triangular cross sectional shape of the continuous portion of an underlying monocrystalline silicon substrate 106 shown in FIG. 1A. In some embodiments, the oxidized surface layer 108 does not penetrate far enough into the monocrystalline silicon surface to form a triangular shape in cross section. In some embodiments, the monocrystalline silicon surface is not thermally oxidized, although some native oxide may form thereon.

The common chamber 104 is separated from the two reservoirs 102 a, b by a membrane layer 112 having two nanoscale openings, here a first nanopore 114a and a second nanopore 114b, formed therethrough. The first nanopore 114a is disposed through a portion of the membrane layer 112 which separates the first reservoir 102a from the common chamber 104. The second nanopore 114b is disposed through a portion of the membrane layer 112 which separates the second reservoir 102b from the common chamber 104, and the divider wall separates the first and second reservoirs 102 a, b from each other.

Source electrodes 116 a, b, disposed in each of the fluid reservoirs 102 a, b, respectively, and a common ground electrode 118 disposed in the common chamber 104, are used to apply independent voltage potentials to each of the fluid reservoirs 102 a, b V₁, V₂ as compared to the ground potential of the common chamber to facilitate co-capture of a single biopolymer strand 120. Once co-capture of the biopolymer strand 120 is achieved by the first and second nanopores 114 a, b, application of competing voltages across the first and second nanopores 114 a, b, i.e., between their electrodes 116a, b and the common ground electrode 118 respectively, are used to create a tug-of-war on the biopolymer strand as it travels from the first reservoir 102a to the second reservoir 102b. Ionic current flows are independently measured through each of the nanopores 114 a, b and the resulting current signal patterns can be used to determine a sequence of the monomer units of the biopolymer strand.

FIG. 1B schematically illustrates trapezoidal cross-section shaped openings 121 formed in a monocrystalline silicon substrate 122 using an anisotropic etch process and a patterned mask layer 128 disposed on the surface thereof. The anisotropic etch process uses inherently differing etch rates for the silicon material of the substrate as between {100} plane surfaces 124 and {111} plane surfaces 126 thereof when exposed to an anisotropic etchant. The actual differing etch rates of the silicon substrate 122 into {100} plane surfaces 124 and {111} plane surfaces 126 depend on the concentration of the etchant in the aqueous solution, the temperature of the aqueous solution, and a concentration of the dopant in the substrate (if any).

In some embodiments, the etching process is controlled to where the etch rates of the {111} plane surfaces 126 and the {100} plane surfaces have a ratio between about 1:10 and about 1:200 such as between about 1:10 and about 1:100, for example between about 1:10 and 1:50, or between about 1:25 and 1:75). Examples of suitable anisotropic wet etchants herein include aqueous solutions of potassium hydroxide (KOH), ethylene diamine and pyrocatechol (EPD), ammonium hydroxide (HN₄OH), hydrazine (N₂H₄), or tetra methyl ammonium hydroxide (TMAH).

Typically, a {100} plane at the surface of monocrystalline silicon substrate will meet the {111} plane in the bulk of the substrate to form an angle α of 54.74°. Thus, in embodiments set forth herein sidewalls defining anisotropically etched openings in a monocrystalline silicon substrate will form an angle with a plane of the field surface of the substrate of about 54.74°.

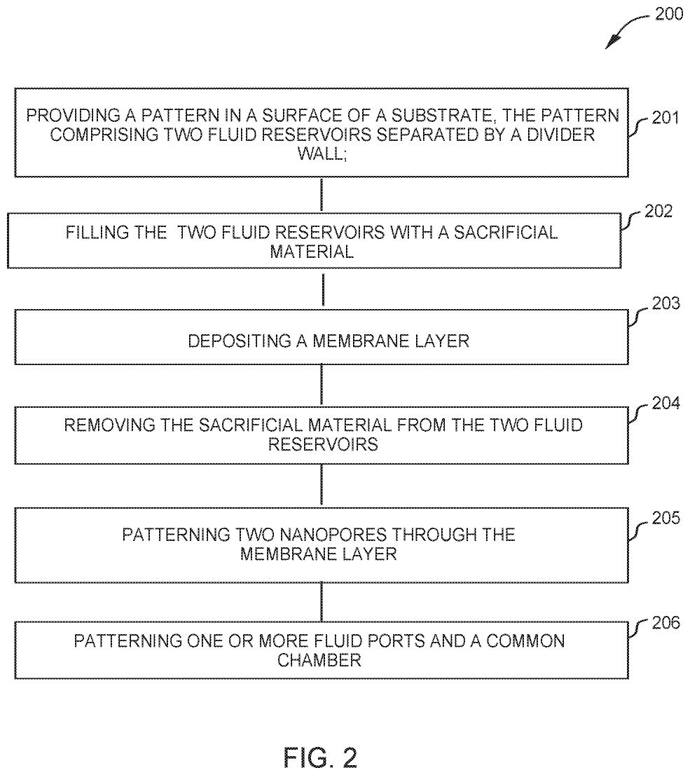

FIG. 2 is a flow diagram setting forth a method of forming a dual pore sensor, according to one embodiment. FIGS. 3A-3L schematically illustrate various activities of the method 200, according to one or more embodiments.

At activity 201 the method 200 includes providing a pattern in a surface of a substrate. Here, the pattern features two fluid reservoirs recessed from a field of the surface, where the two fluid reservoirs are separated by a barrier wall formed of non-recessed or partly recessed portion of the substrate. In one embodiment, providing the pattern in the surface of the substrate surface includes forming a patterned mask layer on the surface of a substrate and transferring the pattern of the etch mask to the underlying substrate surface using an anisotropic etch process. FIGS. 3A and 3B illustrate a substrate 302 having a patterned mask layer 304 disposed thereon. FIG. 3A is a schematic plan view of the substrate and mask thereover. FIG. 3B is a sectional view of a portion of FIG. 3A taken along line A-A.

Here, the patterned mask layer 304 is formed of a material which is selective to anisotropic etch compared to the underlying monocrystalline silicon substrate. Examples of suitable mask materials include silicon oxide (SiₓO_{y}) or silicon nitride (SiₓN_{y}). Herein, the mask layer 304 has a thickness of about 100 nm or less, such as about 50 nm or less, or about 30 nm or less. The mask layer 304 material here is patterned using any suitable combination of lithography and material etching patterning methods. The pattern features a first opening 306a and a second opening 306b disposed through the mask layer 304 which are spaced apart from one another to define a mask wall 308 disposed therebetween. Here, openings 306 a, b define two sides of a recessed pattern generally surrounded by the masking material and divided by the mask wall 308, and individual generally circularly cylindrical islands 310 of mask material interspersed in the respective recess.

In FIG. 3A the two openings 306 a, b form a generally symmetrical “H” shaped pattern which is bifurcated by the mask wall 308. In other embodiments, the pattern may be any suitable symmetrical or asymmetrical shape for example an “X” shaped pattern, a “+” shaped pattern, a “K” shaped pattern, or any other desired pattern where the to-be-formed reservoirs will come into close proximity to form the a divider wall having a desired width.

In FIG. 3B the islands 310a are bisected by line A-A are shown in cross section, the islands 310b are behind the section defined by line A-A. A width X₁ of the mask wall 308 at the field (upper) surface of the substrate 302 and the amount of material removed from the 111 plane during a subsequent anisotropic etch process determines the minimum spacing between the two nanopores of the dual nanopore sensor. Here, the width X₁ is less than about 300 nm, such less than about 250 nm, less than about 200 nm, or for example less than about 180 nm. The mask layer 304 further includes a plurality of discontinuous features as individual mesas or islands 310 of mask material, distributed within the boundaries defined by the walls of each of the openings 306 a, b.

Transferring the mask pattern to the surface of the substrate 302 typically comprises anisotropically etching the monocrystalline silicon thereof by exposing the field surface thereof to an etchant through the openings 306 a, b of the mask layer 304. In one embodiment, anisotropically etching the substrate 302 comprises exposing the substrate surface to an anisotropic wet etchant to form first and second reservoirs 312 a, b (shown in FIGS. 3C-3D) each having a base surface which is recessed from the field surface of the substrate to a desired depth D. Here, each of the first and second reservoirs 312 a, b will form a respective fluidly connected volume in the resulting dual nanopore sensor. After the substrate surface is patterned, the mask layer 304 may be removed therefrom using any suitable method, such as by exposure to an aqueous solution of phosphoric acid.

FIG. 3C is a schematic plan view of the patterned surface of the substrate 302 mask layer 304 is removed. FIG. 3D is a schematic sectional view of FIG. 3C taken along line B-B. Here, the patterned surface of the substrate 302 features the two fluid reservoirs 312 a, b, which are spaced apart from one another by a divider wall 314. The fluid reservoirs 312 a, b each have a maximum depth D₁ of measured in a direction orthogonal the field surface of the substrate 302. Typically, the maximum depth D₁ is more than 0.1 μm, such as more than 0.5 μm, or more than about 1 μm, for example between about 0.5 μm and about 2 μm. Here, the patterned surface further includes a plurality of support structures 316 corresponding to the locations of the plurality of islands 310 described above. Each of the plurality of support structures 316 have a truncated cone or pyramidal shape which forms a trapezoidal shape in cross-section where the field surfaces of the support structures 316 are narrower than the bases thereof. Here, the widths W₂ of individual support structures 316 at the field surfaces thereof are in a range of between about 0.1 μm and about 5 μm, such as between about 0.5 μm and about 2.5 μm. Individual ones of the plurality of the support structures 316 spaced apart from the walls of first and second openings 306 a, b and from one another by a distance suitable for supporting portions of a to-be-formed membrane layer which will span the reservoirs 312 a, b. In some embodiments, the support structures have a center to center spacing of 10 μm or less, such as about 7.5 μm or less, or for example about 5 μm or less.

Here, the divider wall 314 has a trapezoidal shape in cross section such that opposing surfaces thereof are sloped to form an angle α of 54.74° with a plane of the field surface of the patterned substrate 302. The width W₁ of the divider wall 314 at the field surface of the substrate 302 is about 200 nm or less, such as 180 nm or less, about 160 nm or less, about 140 nm or less, about 120 nm or less, or about 100 nm or less. In some embodiments, the width W₁ is in the range between about 60 nm and about 140 nm, such as between about 80 nm and about 120 nm. In other embodiments the openings forming the fluid reservoirs 312 a, b, are etched until the divider wall 314 has a triangular shape in cross section.

Here, the method 200 further includes forming a dielectric layer on the patterned surface of the substrate 302 by one or both of thermally oxidizing the monocrystalline silicon surface or by depositing a dielectric material thereon. For example, in some embodiments, the method 200 further includes thermally oxidizing the surface of the substrate to form an oxide layer, herein the first dielectric layer 318 (shown in FIG. 3E). In some embodiments, the silicon surface is oxidized to provide a first dielectric layer 318 having a thickness of more than about 5 nm, such as more than about 10 nm, more than about 20 nm, or more than about 30 nm. In some embodiments, the silicon surface is oxidized to provide a first dielectric layer 318 having a thickness of between about 20 nm and about 80 nm. Typically, thermal oxidation comprises exposing the substrate 302 to steam or molecular oxygen (O₂) in a furnace at a temperature between about 800° C. and about 1200° C. Because thermal oxide incorporates silicon consumed from the substrate 302 with supplied oxygen, about 44% of the thickness of the first dielectric layer 318 will lie below the original silicon surface and about 56% of the thickness of the first dielectric layer 318 will extend thereabove. Thus, thermally oxidizing the silicon surface to form the first dielectric layer 318 will increase the width of the wall by more than about 1.12 times the thickness of the resulting thermal oxide. In some embodiments, the silicon surface is thermally oxidized to a depth where the portion forming the divider wall has a triangular cross sectional shape. In some embodiments, the silicon surface is thermally oxidized to a depth where the portion forming the divider wall maintains its trapezoidal cross sectional shape.

In some embodiments, the method 200 includes depositing a dielectric material, such as the second dielectric layer 320 (FIG. 3E) on the patterned surface to cover and thus line the surfaces of the two fluid reservoirs 312 a, b and the field. Here, the second dielectric layer 320 comprises any suitable dielectric material, such as a silicon oxide (SiₓO_{y}), a silicon nitride (SiₓN_{y}), a silicon oxynitride (SiOₓN_{y}), or an oxide, nitride, or oxynitride of Group III, Group IV, Lanthanide series elements, combinations thereof, or layered stacks of two or more thereof. For example, in some embodiments, the second dielectric layer 320 comprises aluminum oxide (Al₂O₃), aluminum nitride (AlN), titanium oxide (TiO), titanium nitride (TiN), tantalum oxide (Ta₂O₅), tantalum nitride (TaN), or combinations thereof. In some embodiments, the second dielectric layer 320 comprises amorphous silicon.

Beneficially, the second dielectric layer 320 prevents or substantially reduces charges from accumulating in the monocrystalline silicon substrate 302 during high frequency nucleotide detection. Thus, the second dielectric layer 320 substantially reduces undesirable background noise to improve the detection resolution of the dual pore sensor. Here, the second dielectric layer 320 is deposited to a thickness of less than about 100 nm, such as less than about 80 nm, less than about 60 nm, or for example between about 20 nm and about 100 nm. Depositing the second dielectric layer 320 increases the width of the wall by more than about 2 times the thickness of the second dielectric layer 320.

Typically, the sloped surfaces of the first or second dielectric layer 318, 320 disposed on opposing sides of the divider wall 314 will form an angle Θ with the plane of the field surface of the substrate 302 having one or both of the dielectric layer 318, 320 disposed thereon. Here, the angle Θ may be the same as the angle α of about 54.74° or may vary to account for non-uniform oxidation of the substrate 302 to form the first dielectric layer 318 and, or, non-conformal deposition of the second dielectric layer 320. For example, in some embodiments the sloped surfaces of the first or second dielectric layer 318, 320 form an angle Θ in a range of about 54.74°+/−5°, or about 54.74°+/−2.5°, or about 54.74°+/−1°.

The second dielectric layer 320 may serve as a CMP stop layer in subsequent planarization operations and, or electrically insulate conductive fluid in the fluid reservoirs 312 a, b from the monocrystalline silicon substrate 302 disposed therebelow. In some embodiments, the method 200 includes one but not both of oxidizing the patterned surface of the substrate 302 to form the first dielectric layer 318 or depositing the second dielectric layer 320. For example, in some embodiments, the patterned surface of the monocrystalline silicon substrate 302 is not thermally oxidized before the second dielectric layer 320 is deposited thereon, although at least some native oxide growth is to be expected. In embodiments that do not include depositing the second dielectric layer 320, the first dielectric layer 318 may serve as a CMP stop layer in a subsequent planarization operation.

At activity 202 the method 200 includes filling the two fluid reservoirs 312 a, b with a sacrificial material 322. In some embodiments, filling the two fluid reservoirs 312 a, b, with a sacrificial material 322 includes depositing a layer of sacrificial material 322 onto the patterned substrate 302, e.g., onto the first dielectric layer 318 or the second dielectric layer 320 (FIG. 3F). In those embodiments, the method further includes removing the sacrificial material 322 from over a field surface of the second dielectric layer 320 (FIG. 3G) to leave the portions of the second dielectric layer 320 over each of the dividing walls exposed. Typically, removing the sacrificial material 322 from the field surface of the second dielectric layer 320 comprises planarizing a surface of the substrate using a chemical mechanical planarization (CMP) process. The planarized surface of the substrate, including the planarized surfaces of the sacrificial material 322 disposed in the fluid reservoirs 312 a, b (shown in FIG. 3E), will provide structural support for a subsequently deposited membrane layer. A suitable sacrificial material will have a high etch rate and CMP removal rate selectivity to the underlying second dielectric layer 320 and a high etch rate selectivity to the material of the membrane layer 112 to-be-formed thereover. Examples of suitable sacrificial materials include phosphosilicate glass (PSG), borophosphosilicate glass (BPSG), polysilicon, amorphous Si, aluminum, carbon-based films, and polymers such as polyimide.

At activity 203 the method 200 includes depositing a membrane layer 324. Here, the membrane layer 324 is deposited onto the field surface of the second dielectric layer 320 and onto the planarized sacrificial material 322 disposed in the fluid reservoirs 312 a, b. In some embodiments, the membrane layer 324 is formed of silicon nitride. In other embodiments, the membrane layer is formed of another suitable dielectric material, such as any of the materials set forth above as suitable for the second dielectric layer 320. Typically, the membrane layer 324 is deposited to a thickness of less than about 200 nm, such as less than about 100 nm, less than about 60 nm, for example less than about 50 nm, or between about 10 nm and about 50 nm, such as between about 20 nm and about 40 nm.

At activity 205 the method 200 includes removing the sacrificial material 322 from the two fluid reservoirs 312 a, b. In one embodiment, removing the sacrificial material 322 includes patterning the membrane layer 324 to form a plurality of vent openings 326 therethrough and removing the sacrificial material 322 through the plurality of vent openings 326. The membrane layer 324 may be patterned using any suitable combination of lithography and material etching patterning methods such as forming a patternable mask layer over the membrane layer 324, patterning the mask layer to form openings corresponding in size and location to the locations of the vent openings 326 using photolithographic techniques, and then etching the portions of the membrane layer 324 exposed by the openings through the mask layer to form the vent openings 326 through the membrane layer 324.

Here, individual ones of the plurality of vent openings 326 have a diameter of less than about 500 nm, less than about 100 nm, or for example less than about 50 nm. In some embodiments, individual ones of the plurality of vent openings 326 have a diameter of between about 1 nm and about 500 nm, such as between about 1 nm and about 100 nm, between about 1 nm and about 50 nm, or for example between about 10 nm and about 40 nm. In some embodiments, individual ones of the plurality of vent openings 326 have a center to center spacing from a vent opening 326 disposed adjacent thereto of less than about 500 nm, such as less than about 300 nm, or less than about 100 nm. The plurality of vent openings 326 may from any desirable pattern suitable for venting volatilized or dissolved sacrificial material 322 disposed in the fluid reservoirs 312 a, b therethrough in a subsequent sacrificial material removal step including the irregularly spaced pattern shown in FIG. 3H.

In one embodiment, the sacrificial material 322 is removed through the vent openings 326 using a plasma based dry etch process. For example, in one embodiment the sacrificial material 322 is exposed through the plurality of vent openings 326 to the plasma activated radical species of a suitable etchant, such as the radial species of a halogen based gas, e.g., a fluorine or chlorine based gas. An exemplary system which may be used to remove the sacrificial material 322 from the fluid reservoirs 312 a, b is the Producer® Selectra™ Etch system commercially available from Applied Materials, Inc., of Santa Clara, Calif. as well as suitable systems from other manufacturers.

In another embodiment, removing the sacrificial material 322 includes exposing the sacrificial material 322, through the vent openings 326, to an etchant having a relativity high etch selectivity to the material or materials used to form the second dielectric layer 320 and the membrane layer 324. Examples of suitable etchants include TMAH, NH4OH, aqueous HF solutions, and buffered aqueous HF solutions such as an aqueous solution of HF and NH4F, and anhydrous HF. Etch byproducts are then removed from the fluid reservoirs 322 a, b by rinsing and drying the substrate. In some embodiments the etch byproducts are removed by rinsing the substrate with deionized water before drying the substrate using N₂ gas or an isopropyl alcohol (IPA) and N₂ gas mixture. In other embodiments, such as in embodiments using anhydrous HF, removing remaining etch byproducts includes heating the substrate to a temperature of more than about 100° C. in a vacuum environment of less than about 40 Torr.

At activity 205 the method 200 includes patterning two nanopores 328 a, b through the membrane layer 324. The nanopores 328 a, b may be patterned using any suitable method. In one embodiment, the nanopores 328 a, b are patterned using the same or a similar process to the process used to form the vent openings 326 as described above. For example, in some embodiments, the vent openings 326 and the nanopores 328a, b are formed in the same lithography and material etching sequence. In other embodiments, the vent openings 326 and the nanopores 328 a, b are formed in sequential lithography and material etching sequences of any order. In other embodiments, the nanopores 328 a, b are formed in a lithography and material etching sequence which is separated from the lithography and material etching sequence used to form the vent openings 326 by another processing operation. For example, in some embodiments the nanopores 328 a, b are formed after the sacrificial material 322 is removed through the vent openings 326 or after a common chamber is patterned as described in activity 206 below.

Here, the two nanopores 328 a, b are formed through respective portions of the membrane layer 324 disposed over each of the fluid reservoirs 312 a, b and thus are positioned on either side of the divider wall 314 proximate thereto. Typically, each of the nanopores 328 a, b have a diameter of less than about 100 nm, such as less than about 50 nm between about 0.1 nm and about 100 nm, or between about 0.1 nm and about 50 nm. Here, the nanopores 328 a, b are spaced apart from one another by a distance X₂ of less than about 600 nm, such as less than about 550 nm, less than about 500 nm, less than about 450 nm, less than about 400 nm, or in some embodiments, less than about 300 nm.

At activity 206 the method 200 includes patterning one or more fluid ports 338 and a common chamber 334 (FIG. 3J). In one embodiment, patterning the one or more fluid ports 338 and the common chamber 334 forming openings in an overcoat layer 330 disposed on the patterned membrane layer 324. Here, the overcoat layer 330 seals the vent openings 326 in the membrane layer 324 where fluid access to the reservoirs 332 a, b disposed therebeneath is not desired. The one or more fluid ports 338 provide fluid access to the fluid reservoirs 332 a, b to facilitate the introduction of electrolytic fluid and biopolymer samples thereinto. The overcoat layer 330 may be formed using any suitable material and method which minimizes penetration of the overcoat material into the vent openings 326. Thus, the material and method chosen to deposit the overcoat layer 330 should prevent undesirable filling of the fluid reservoirs 332 a, b therewith through the vent openings 326.

In one embodiment, the overcoat layer 330 is formed by spin coating a polymer precursor onto the patterned membrane layer 324 and curing the polymer precursor by exposure to thermal or electromagnetic radiation. In some embodiments, the fluid ports 338 and the common chamber 334 areas are then etched through the cured polymer using a lithography-etch processing sequence. In other embodiments, the polymer precursor is photosensitive, such as a photosensitive polyimide precursor or benzocyclobutene (BCB), and the desired pattern is exposed directly thereon. Unexposed photosensitive polymer precursor is then removed from the substrate to form the fluid ports 338 and the common chamber 334 areas. Herein, the fluid port 338 and the common chamber 334 areas may be formed at the same time, sequentially, or in processing operations separated by intervening processing activities.

In another embodiment, the overcoat layer 330 comprises a polymer film layer, such as a polyimide film, which is laminated onto the surface of the membrane layer 324 before or after the fluid port 338 and the common chamber 334 areas are formed (patterned) therethrough.

FIG. 3J is a schematic plan view of a dual pore sensor 300, formed according to embodiments described herein, which may be used in place of the dual pore sensor 100 described in FIG. 1A. FIG. 3K is a sectional view of a portion of FIG. 3J taken along line D-D. Here, the dual pore sensor 300 features a patterned substrate 301 and the membrane layer 324 disposed on the patterned substrate 301. The pattern includes two recessed regions separated by the divider wall 314. Each of the two recessed regions have one or more base surfaces 303 which are substantially parallel to a plane of the field (upper) surface of the patterned substrate 301. The base surfaces 303 and one or more sidewalls 305 of the each of the recessed regions (shown in phantom in FIG. 3J), the membrane layer 324, and the divider wall 314 (having one or both dielectric layers 318, 320 disposed thereon) collectively define the first fluid reservoir 332a and the second fluid reservoir 332b respectively.

Here, the membrane layer 324 is spaced apart from the one or more base surfaces 303 of the recessed regions by a distance D₂ of more than about 0.5 μm, such as more than about 1 μm, more than about 1.5 μm, or more than about 2 μm. The surfaces of the recessed regions and the divider wall 314 are lined with one or both of the first or second dielectric layer 318, 320. A first nanopore 328a is disposed through a portion of the membrane layer 324 disposed over the first fluid reservoir 332a and a second nanopore 328b is disposed through a portion of the membrane layer 324 disposed over the second fluid reservoir 332b. In some embodiments, membrane layer 334 has a plurality of vent openings 326 formed therethrough which are sealed with a overcoat layer 330 disposed thereover. The overcoat layer 330 includes openings disposed therethrough to define the common chamber 334 and the one or more fluid ports 338 disposed over each of the respective fluid reservoirs 332 a, b. The common chamber 334 is in fluid communication with each of the fluid reservoirs 332 a, b, through respective nanopores 328 a, b.

Here, the reservoir facing surface of the membrane layer 324 is substantially planer and is parallel to the field surface of the patterned substrate 301. In some embodiments, the membrane layer 324 is spaced apart from the base surfaces 303 of the recessed regions by the plurality of support structures 316 (and the dielectric liner disposed thereon). Typically, individual ones of the plurality of support structures 316 have a trapezoidal shape in cross section. For example, herein surfaces of the one or both of the plurality of support structures 316 and the divider wall 314 are sloped to form an angle Θ with a reservoir 332 a, b, facing surface of the membrane layer 324 of less than 90°, such as less than about 60°, or with the range of about 54.74°+1-5°, or about 54.74°+1-2.5°, or 54.74°+1-1°, for example about 54.74°.

In some embodiments, a ratio of the depth D₂ of the recessed regions to the nanopore spacing X₂ (described in FIG. 3I) is more than about 1:1, such as more than about 2:1, more than about 3:1, more than about 4:1, or for example more than about 5:1. Here, the depth D₂ is measured from a plane of the field surface of the patterned substrate 301 to the base surfaces 303 of the fluid reservoirs 312 a, b, i.e., the distance between reservoir facing surfaces of the membrane layer 324 and the base surfaces 303. In some embodiments, the dual pore sensor 300 further includes electrodes disposed in each of the fluid reservoirs 332 a, b and the common chamber 334, such as the electrodes 116 a, b and 118 described in FIG. 1A.

In some embodiments, the method 200 further includes forming a vent opening extension layer 332 (shown in FIG. 3L) on the membrane layer 324 before removing the sacrificial material 322 from the fluid reservoirs at activity 208. Forming the vent opening extension layer 332 before removing the sacrificial material 322 may prevent damage to, e.g., collapse of, the fragile underlying membrane layer 324 when the overcoat layer 330 is formed thereon. In those embodiments, the vent opening extension layer 332 may be formed of the same material and methods which are suitable for forming the subsequent overcoat layer 330 and are set forth in the description of activity 208. Once the vent opening extension layer 332 is deposited onto the membrane layer a plurality of openings 340 are formed therethrough. Each of the plurality of openings 340 are coaxially disposed and/or in fluid registration with a corresponding vent opening extension layer 332 opening 326 in the membrane layer 324. Examples of suitable methods of forming the plurality of openings 340 include lithography-etch processing sequences and direct exposure of a photosensitive polymer precursor in embodiments where the opening extension layer 332 is formed therefrom. In embodiments which include the vent opening extension layer 332 one or both of the fluid ports and common chamber opening are further formed through the vent opening extension layer to expose the membrane layer disposed there beneath.

In some embodiments, the dual pore sensor 300 described in FIGS. 3J-3K further includes the vent opening extension layer 332 described above in FIG. 3L.

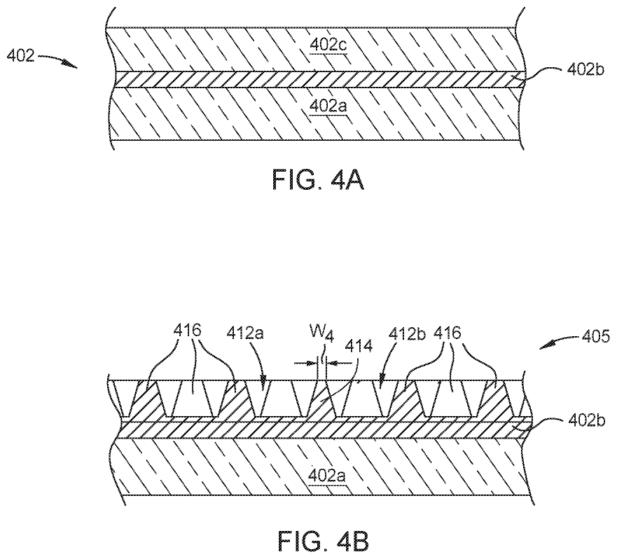

In another embodiment, the substrate is a silicon on insulator (SOI) substrate 402 (shown in FIG. 4A) featuring first and second (monocrystalline) silicon layers 402a and 402c and an electrical insulator layer 402b, such as a sapphire layer or a silicon oxide layer (SiₓO_{y}), interposed therebetween. In this embodiment, the surface of the substrate 402, i.e. the second silicon layer 402c, is patterned using one or a combination of embodiments of the method 200 set forth above to form a patterned substrate 405 (FIG. 4B). The pattern comprises two fluid reservoirs 412 a, b, a divider wall 414 having a width W₄ at the field surface thereof, and a plurality of structural supports 416 formed in the second silicon layer 402c. The patterned second silicon layer 402c is thermally oxidized to the depth of the electrical insulator layer 402b disposed there beneath and a dual pore sensor may be formed therefrom using activities 202-208 of the method 200 or alternative embodiments thereof.

In some embodiments, the method 200 above includes forming the pattern in the second silicon layer 402c and thermally oxidizing the second silicon layer 402c to the depth of the electrical insulator layer 402b. In some embodiments, the patterned second silicon layer 402c is not oxidized to the depth of the electrical insulator layer 402b. For example, in some embodiments the patterned second silicon layer 402c is thermally oxidized to a depth of less than about 100 μm, such as less than about 50 μm, less than 25 μm, or for example less than about 10 μm.

In some embodiments, the dual pore sensor 300 described in FIGS. 3J-3K features one or both of the patterned substrate 405 in place of the patterned substrate 301 and the vent opening extension layer 332. In some embodiments, the patterned substrate 405 further includes a dielectric liner, such as the second dielectric 320 described above, deposited thereon.

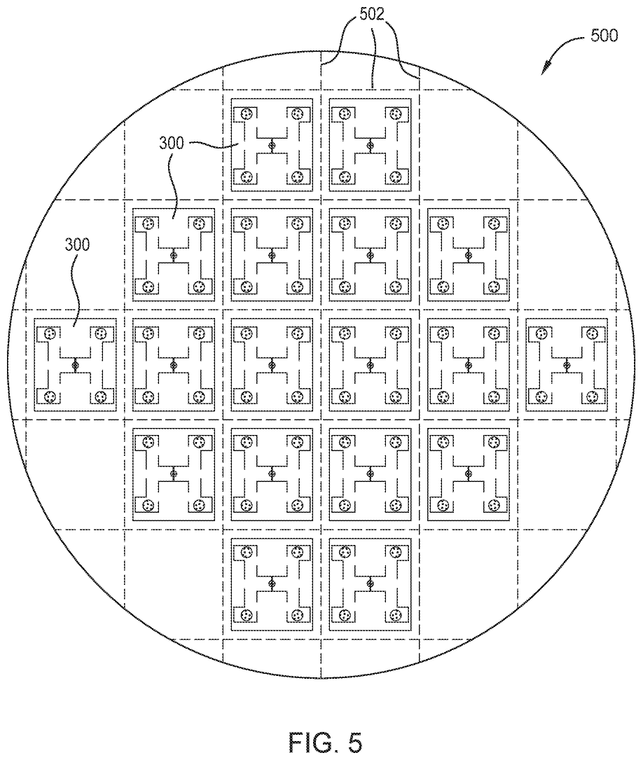

Typically, the methods provided herein are used to simultaneously manufacture a plurality of dual pore sensors on a single substrate, such as the single wafer substrate 500 shown in FIG. 5. The wafer substrate 500 is then singulated into individual dies to provide a plurality of dual pore sensors 300.

Exemplary dimensions of a sensor 300 formed using the methods set forth herein is less than about 20 mm per side, such as less than about 15 mm, or less than about 10 mm, or for example between about 1 mm and about 20 mm. In some embodiments a width of a singulated sensor formed using the embodiments set forth herein is between about 1 mm and about 100 mm.

The dual pore sensors provided herein may include any one or combination of the features described above in FIGS. 1A, 3J-3K, 3L, and 4B, including alternate embodiments thereof. The dual pore sensors provided herein may be singulated or may comprise one of a plurality of dual pore sensors formed on a single wafer substrate, such as the single wafer substrate 500 described in FIG. 5.

Beneficially, the methods described herein allow for high volume manufacturing, and improved quality, repeatability, and manufacturing costs of a dual pore sensor. Further, the manufacturing methods described allow for interpore spacing of 300 nm or less to beneficially increase the number of relativity shorter biopolymer strands which may be sequenced using a dual pore sensor.

While the foregoing is directed to embodiments of the present disclosure, other and further embodiments of the disclosure may be devised without departing from the basic scope thereof, and the scope thereof is determined by the claims that follow.

1. A method of forming a dual pore sensor, comprising:
providing a pattern in a surface of a substrate, the pattern comprising two fluid reservoirs separated by a divider wall;
depositing a layer of sacrificial material into the two fluid reservoirs;
depositing a membrane layer;
patterning two nanopores through the membrane layer;
removing the sacrificial material from the two fluid reservoirs; and
patterning one or more fluid ports and a common chamber.

2. The method of claim 1, wherein the pattern further comprises a plurality of support structures disposed within respective boundaries defined by walls of the fluid reservoirs.

3. The method of claim 2, wherein individual ones of the plurality of support structures have a trapezoidal shape in cross-section.

4. The method of claim 1, wherein the substrate comprises monocrystalline silicon.

5. The method of claim 4, wherein the patterned surface of the substrate comprises a layer of thermally oxidized silicon.

6. The method of claim 4, wherein the patterned surface of the substrate comprises a layer of deposited dielectric material.

7. The method of claim 4, wherein opposing surfaces of the divider wall are sloped to each form an angle with a plane of a field surface of the substrate within a range of 54.74°+/−5°.

8. The method of claim 1, wherein the two nanopores are formed through respective portions of the membrane layer disposed over each of the fluid reservoirs.

9. The method of claim 1, wherein
the substrate comprises a first silicon layer, a second silicon layer, and an electrical insulator layer interposed therebetween,
the pattern is provided in the second silicon layer, and
the method further includes thermally oxidizing at least a portion of the patterned second silicon layer.

10. The method of claim 1, wherein removing the sacrificial material from the two reservoirs comprises patterning a plurality of vent openings through the membrane layer and removing the sacrificial material through the plurality of vent openings.

11. A method of forming a dual pore sensor, comprising:
providing a pattern in a monocrystalline silicon surface of a substrate, the pattern comprising:
two fluid reservoirs separated by a divider wall; and
a plurality of support structures disposed within respective boundaries defined by one or more walls of the two fluid reservoirs;
filling the two fluid reservoirs with a sacrificial material;
depositing a membrane layer;
patterning two nanopores through the membrane layer,
removing the sacrificial material from the two fluid reservoirs; and
patterning an overcoat layer to define one or more fluid ports and a common chamber.

12. The method of claim 11, wherein
the substrate comprises a first silicon layer, a second silicon layer, and an electrical insulator layer interposed therebetween
the pattern is provided in the first silicon layer, and
the method further includes thermally oxidizing at least a portion of the patterned first silicon layer.

13. The method of claim 11, further comprising thermally oxidizing the patterned monocrystalline silicon surface.

14. The method of claim 11, further comprising depositing a layer of dielectric material before filling the two fluid reservoirs with a sacrificial material.

15. A method of forming a dual pore sensor, comprising:
providing a patterned substrate, the pattern comprising:
two fluid reservoirs separated by a divider wall, wherein opposing surfaces of the divider wall are sloped to each form an angle with a plane of a field surface of the substrate within a range of 54.74°+/−5°; and
a plurality of support structures disposed within respective boundaries defined by one or more walls of the two fluid reservoirs, wherein individual ones of the plurality of support structures have a trapezoidal shape in cross section;
filling the two fluid reservoirs with a sacrificial material;
depositing a silicon nitride membrane layer;
patterning two nanopores through the silicon nitride membrane layer,
removing the sacrificial material from the two fluid reservoirs; and
patterning an overcoat layer to define one or more fluid ports and a common chamber.
